(19) United States
(12) Patent Application Publication
(10) Pub. No.: US 2009/0093856 A1
(43) Pub. Date: Apr. 9, 2009

(54) High fidelity electronic tactile sensor and stimulator array, including sexual stimulus

(76) Inventors: Mady Attila, Kihei, HI (US); Radu Elias, Toronto (CA)

Correspondence Address: Attila Mady, 105 Manino Circle, Suite 203, Kihei, HI, 96753, US

(21) Appl. No.: 11/868,365

(22) Filed: Oct. 5, 2007

(51) Int. Cl. A61N 1/00; A61B 5/04; A61B 5/05; A61F 5/00

(52) U.S. Cl. 607/6; 600/372; 600/38; 600/554

(57) ABSTRACT

Modular electronic and/or combined electronic/mechanical apparatus for the detection, analysis, transmittal and delivery of tactile stimuli (touch). Sexual application is suggested.

High fidelity electronic tactile sensor and stimulator array

### TECHNICAL FIELD

Remote, transmission, touch, communications, sexual devices.

### LEXICON

Biological touch receptor is defined as a Meissner corpuscle, Merkel cell, Pacinian corpuscle, Ruffini nerve endings, free nerve endings, muscle spindle tension proprioceptor, temperature sensors and any other sensors known or not known that transduce primarily vibration modalities.

Tactile stimuli are defined as those that would be detected and interpreted by a human being through the skin and through components of the peripheral and central nervous system dedicated to touch, respectively.

Tactile stimuli would include information received through other analogous means, such as tendon, bone and dental vibration reception, auditory ossicle vibration not mediated by the inner ear, sensation from tensor tympani, etc.

The detection component of the Apparatus described in the Claims of this invention is defined as those components dedicated to the detection of mechanical vibrations, whether longitudinal analogous to sound or transverse analogous to ocean waves.

The analysis component of the Apparatus described in the Claims of this invention is defined as those components dedicated to the translation of this information into a set of signals that can be transmitted with sufficient fidelity by the available transmission means and that can delivered appropriately by the available delivery component of the Apparatus described in the Claims of this invention and that would achieve the desired sensation and effect in the target species.

The transmission component of the Apparatus described in the Claims of this invention is defined as those components dedicated to the transmission of information from the detection and analysis components to the delivery component of the Apparatus described in the Claims of this invention. The transmission component of the Apparatus described in the Claims of this invention does not include those components dedicated within each individual subsection to delivery of signal from one component within that stage to another component within the same stage.

The delivery component of the Apparatus described in the Claims of this invention is defined as those components dedicated to delivering an appropriate electronic and/or electromechanical signal with sufficient fidelity to generate a desired sensation and effect in the target species.

The electrode delivery array of the Apparatus described in the Claims of this invention is defined as the aggregate of electrical conduits exiting from the delivery component of the Apparatus described in the Claims of this invention and conducting signals from said component to the desired target surface.

Sexual stimulation is defined not only as the use of the Apparatus described in the Claims of this invention on the subset of target sites that are generally defined as erogenous, but also the use of the Apparatus described in the Claims of this invention on any site if that use would result in any effect on sexual arousal.

A bipolar electrode array is an array of coupled electrodes that, when actuated and provided with a resistor that spans the spatial gap between them, in pairs complete a complete circuit and thus are able to transmit an electric potential and/or current. NOTE: a bipolar array does not have to be physically divided into individual electrode pairs, but is determined by the fact that electrodes are paired individually to complete an electrical circuit.

To further clarify the meaning of the term “bipolar” as used in this application, this term is to mean the opposite of operating in a unipolar configuration with central ground.

(“Central ground” means that multiple electrodes refer to the same ground to complete their circuit, and/or that the reference ground is at a significant geographic distance, and/or that there is a third object (such as a patient's body in the case of cautery) of significant size and impedance interposed between the electrode and reference ground. This construct results in multiple possible conduction pathways and therefore in low fidelity compared to a bipolar arrangement.)

Tactile sensations are not to be restricted to touch, but are also to be understood to subsume sensations such as cold, heat, humidity, elasticity, as well as any other specific information gleaned from the use of apposition of skin to a substrate. Position sense, muscle tension and similar feedback data also falls in this category. In this proposal the terms “tactile sensation” and “touch are to be assumed to be interchangeable and inclusive.

Tachyplaxis is diminished response to successive identical stimuli.

### BACKGROUND OF THE INVENTION

For anyone in doubt of man's infinite drive and profound need to better himself, the reader is referred to extensive prior art regarding various self-stimulation means (i.e.: sexual apparatus). An untold amount of ingenuity and libido has been invested through the ages in every conceivable variation of mechanical means of tactile sensory stimulation.

The need for tactile stimulus in all species is well known. There is a vast body of scientific research that documents the profound impact of touch on the everyday existence of every species. Even bacteria are capable to responding to touch with a number of responses varying from ingestion of material (endocytosis) to secretion (pinocytosis) to even self-destruction. Higher vertebrates have very complex mechanisms of interpreting and responding to touch that govern all modes of existence, including reproduction.

Evidence exists that touch is the most complex and subtle of all sensory imputs in its effects. Touch (and the absence there of) regulates the level of aggression of an animal (including a human), its intra- and interspecies communication (for example, a street cat versus a domesticated cat interacting with a human), as well as an animal's sexual conduct. It is also known that social bonding skills are transmitted to offspring by a mother in large parts through touch.

Adult creatures across many species (including humans) increasingly compartmentalize tactile sensory stimuli into the strictly utilitarian (i.e.: another modality of examining target objects and validating their identity) versus sexual as they age. It is thus not surprising that almost all attempts to date in regards to artificially delivering tactile sensory stimuli have focused on the erogenous zones. Further, all proposals in this area to date (again, the reader is referred to prior art) have concentrated on the delivery of mechanical stimuli, whether generated through electronic, electromechanical or mechanical means.

Only two patents address the possibility of tactile stimuli through purely electronic means (USPTO 6930590 and USPTO #6430450). Only two more (USPTO #4124028 & 4532938) contemplate the possibility of remote transmission and delivery of sexual stimuli of very limited fidelity. Yet, as shall be discussed below, the manipulation and delivery of high-fidelity tactile stimuli through electronic means is likely to be of great utility far beyond the bounds conceived by visionaries restricted to creative deviancy. Further, advances in our understanding of physiology, materials technologies and miniaturization, along with the enormous explosion in signal processing capabilities (Moore's law) have now made such an endeavor feasible.

As mentioned in the claims, the system proposed in the Claims of this patent application consists of detection, analysis, transmission and delivery components. The same is the case with living systems. In the case of human beings, anatomical studies have revealed that the first stage—detection—is accomplished through four different types of mechanoreceptors in the human skin to detect and process tactile stimuli: Meissner corpuscles, Merkel cells, Pacinian corpuscles and Ruffini nerve endings. Meissner corpuscles and Merkel cells detect light touch and are located right beneath the surface of the skin at a depth of less than 1.0 mm. Analogous to piezoelectric crystals, they transduce very slight inputs of mechanical energy into action potentials. Pacinian corpuscles are located deeper in the skin, typically about 2 mm below the skin surface, and respond to strong pressure. Ruffini endings are located around the base of hairs and detect hair movements.

These individual receptors each detect a mechanical (vibratory) stimulus, measure its intensity and then provide a specific response frequency. Note, both vibration frequency and vibration amplitude is coded into frequencies in the nerve (in a manner analogous to an FM—frequency modulation—signal in radio transmission, in contrast to AM—amplitude modulation). These sensors are thus far more than passive electromechanical transducers. They also have some adaptive abilities and compress the detected signal according to a relationship called Weber's Law.

Weber's law describes the correlation of sensory stimuli to sensory response. In the case of the tactile system, however, Weber's Law holds all the way from the individual receptors to the CNS. This is because in the case of tactile stimuli the function of the attached nerves as a whole and the resultant perception in response to stimuli of various intensities is simply a result of the sum of the output of the individual receptors. This is in contrast to the retina, for example, where bipolar, amacrine and horizontal cells perform incredibly complex local signal processing, integration and even rudimentary pattern, shape and movement analysis.

Weber's law is well validated and states that stimuli generate responses in proportion to the natural log of the increase of the stimulus. Specifically, Weber's law states that

dR=k dS/S

(where R stands for response, S stands for stimulus and k is a constant specific to the particular sense modality). In the tactile sensory system each type of sensor has a different k (constant). Each individual sensor is also capable of being inhibited through prior stimulus (called tachyplaxis).

As mentioned above, these signals are then lumped together into the nerve, resulting in an overall response frequency. Sensory frequency is thus converted into an electrical signal of a relatively constant amplitude but of a very specific frequency pattern, which is really an aggregate of the input of the individual receptor contributions. Thus, for instance, the contribution of one thousand Pacinian corpuscles might result in In 1000, or approximately 9 times, the frequency of a single Pacinian corpuscle.

The nerves integrate these resultant signals into a specific sequence of frequencies. These signals are then sent to the dorsal column, through the ascending columns to the thalamus and then the areas of the parietal lobes dedicated to touch sensation. Tactile signals from bone vibration receptors, periosteal vibration receptors and tendon receptors are also mapped this way.

Injurious stimuli such as extreme heat or cold are detected by an independent mechanism, namely free nerve endings. They are mapped independently into a different area of the spinal cord, follow the spinothalamic tract and are processed in a separate area of the thalamus. Tactile pain signals from bone, periosteum and all signals from synovium are mapped this way.

Dental vibration is sensed separately and mapped through the Trigeminal nerve directly into the CNS (the mesencephalic ganglion). Sensation from the face, tongue and the rest of the mouth is delivered through various cranial nerves.

The actuator apparatus of a human being is the utilization of tactile information to effect physical changes on the surrounding environment. This includes other human beings and, more specifically, sexual situations. These functions, as well as touch sensations from inanimate objects, can all be emulated through electronic means.

It should obvious from the above description that the full tactile system in living systems (particularly in higher vertebrates) is exceedingly complex and probably not amenable to exact duplication. A thorough understanding of the basic principles of its function does, however, permit construction of an analogous system that can be achieved with current tools and can deliver a particular pattern of tactile information to the CNS that would result in the same perception as if the recipient had experienced the original raw touch stimulus that is encoded (“virtual reality”).

As with the original living organism, the complete sequence of touch detection to touch delivery must begin with detection. The basic physics of waves and recent advances in materials technologies have turned the requisite precision in signal detection and processing into a trivial challenge. The two types of signals (longitudinal, meaning rarefactions and densifications along the axis of wave propagation such as sound and transverse, an oscillation perpendicular to the axis of propagation, such as waves at a mixed phase interface, or electromagnetic waves) can both be detected through direct means (such as application of piezoelectric crystal array or the use of electroactive polymers as described in USPTO #6809462 directly to an interface to detect vibration) or indirect means (such as laser interferometry or other electromagnetic contouring or other remote contour sensing means to detect minute variations in the contours of an object).

Encoding of these signals involves some estimates. Based on Weber's law, a basic knowledge of individual receptor function (including the individual constants of the logarithmic compression) and density of the individual receptors in any given area of the body, a simple numerical computation will estimate the response that would be generated at each individual point of the detector array (i.e.: the surface where the original signal was generated). This signal is then stored for later transmission and direct delivery through the proposed bipolar electrode delivery array. Sufficient fidelity in transmission and delivery shall result in a perception identical to that which would have been experienced by the subject if they had been exposed to the original stimulus.

It takes few such calculations to realize just how limited the necessary amount of information is to achieve the requisite fidelity. This is because—particularly given the incredible amount of information we glean from touch and the absolute confidence we place in it—the resolution of our touch senses is surprisingly limited. Two point discrimination in less sensitive areas of the human body can be as much as 1 cm (on the back). It is much less than that in most animal species. Conversely, fine discrimination in the fingertips, labia and erogenous areas (including the genitals) is well circumscribed and predictable. Even in these most sensitive areas information density is orders of magnitude less than for vision or hearing.

In fact, this is probably the secret of touch's reliability. Other methods of sensory stimulus can be very readily overwhelmed with sensory overload. Touch abstracts the essentials and discards the rest. Yet, as mentioned, even touch can be overwhelmed, particularly by tactile stimuli that overload receptors. This leads us to one more remarkable effect of electronic tactile sensory stimulus, namely its immunity to tachyplaxis. Since primary attenuation of tactile stimuli occurs at the receptor level, the central nervous system attenuates signals much less than with other senses and tachyplaxis to non-mechanical stimuli does not occur (unless they are so intense that they damage the sensors or nerves).

### SUMMARY OF THE INVENTION

The present invention relates to the remote transmission of tactile stimuli through the use of modular electronic and/or combined electronic/mechanical apparatus for the detection, analysis, transmittal and delivery of tactile stimuli (touch). Sexual application is suggested.

### DETAILED DESCRIPTION OF THE INVENTION

The first component of the Apparatus described in the Claims of this invention is the vibratory stimulus detection and analysis component. Vibration in the target is detected either by direct means, or through laser interferometry. This information is gathered either simultaneously at multiple loci, in an individualized pattern to cover areas of maximal interest, or in a standard grid raster scanning pattern analogous to the acquisition of video information.

The gathered information is processed into a matrix of surface frequency patterns for each locus. This matrix of patterns in turn is translated into a matrix of electrical signals that would be necessary to replicate the effect in the target subject. This is achieved in a manner analogous to a limited Fourier transform as follows:

As a for instance, take a matrix of 10×10 loci (no images are provided since this is a standard concept). Take locus (1,1) of this matrix and assume that 1 Hz is required to generate the requisite effect. If locus (1,2) experiences two times the deflection of locus (1,1) (and, thus, two times the intensity of stimulus), application of Weber's law will result in the equation:

Y=k In 2+0

(where Y is the required delivered signal frequency

2 is the ratio of the intensity of the new stimulus compared to the old and k is a known constant)

The physical dimensions of individual loci will depend on the area of the body intended to be stimulated. The physical dimensions of individual loci shall correspond to limits of two point resolution. The individual signals to be transmitted and delivered will be calculated based on the original signal and knowledge of the approximate number of each of the four types of receptors in the individual locus of the target area (for instance, there might be 600 Meissner, 4000 Pacinian, 380 Merckel and 800 Ruffini receptors in a given specific locus). Since the response of each type of receptor to a given pattern of amplitudes and frequencies is well characterized, it will be a trivial matter to calculate these responses and then sum them together to be delivered at the desired time through the delivery array.

The transmission component of the apparatus can either link the vibratory signal detection and analysis component with the delivery component, or the source signal synthesizer with the delivery component. Source signal synthesizer can be a standard signal generator, a computer program generating a modulated signal, or in the preferred embodiment a small central processing microprocessor integrated into the signal delivery component.

Of note, the initial embodiment of the Apparatus described in the Claims of this invention is envisioned to occur without this detection and encoding component. Electrical signals of sufficient fidelity and variety can be generated through other paradigms to provide an appropriate sensory stimulus when delivered through the electrode delivery array. Particularly in the area of sexual stimulus, signals generated independent of originating real life physical vibratory stimuli are envisioned (ab initio or de novo synthesized signals, such as a simple sine wave of a certain frequency).

The essential and indispensable portion of the Apparatus described in the Claims of this invention therefore consists of the signal delivery component itself. This component would be equipped to accept signals, would contain a microprocessor to process and temporally parse this signal, a multiplexer to distribute this signal and a bipolar electrode array to deliver the signal to the skin.

The entire desired coded signal batch can either be delivered to the device during the time of manufacture as a non-addressable set of instructions permanent to the CPU, or can be communicated through an input port through a physical connection (standard, such as FireWire, or non-standard) or wireless means. In the latter case the CPU of the device or apparatus would have certain protected data architecture analogous to BIOS and not accessible to the user.

The data signal can either be administered as an immutable sequence (envisioned in earlier embodiments), versus a data stream that would be amenable to certain levels of modulation via a remote control or direct user interface via the aforementioned (hardwired or wireless) input connections. It is envisioned that users of these devices will enthusiastically share information and specific data streams to control each other's devices.

The signal is parsed by the CPU according a certain temporal sequence to be (either predetermined, programmable or controllable by means of a remote control device) into the input channels of a multiplexer. An analog to analog multiplexer is preferred due to its lower complexity, but a digital multiplexer may be utilized with appropriate input and output analog to digital and digital to analog converters as indicated. The multiplexer is directed through an appropriate command signal stream to deliver the stimulus signal to an appropriate sized array of bipolar electrodes in the appropriate spatial sequence either to reflect the original stimulus to be delivered, or to achieve the desired effect (in the case of sexual stimuli).

The signal is delivered to the end user by means of an array of bipolar electrodes. The array must be bipolar in order to define a specific stimulus location, as a centrally ground¹ array would lack adequate delivered stimulus resolution and definition.² However, this is not to say that only electrodes within a specific bipolar coupling would be used to define a signal delivery locus. Electrodes of spatially distant electrode pairs would also be able to be coupled together, but always with the intent of forming a stimulus to a defined and known area. ¹ “Central ground” means that multiple electrodes refer to the same ground to complete their circuit, and/or that the reference ground is at a significant geographic distance, and/or that there is a third object (such as a patient's body in the case of cautery) of significant size and impedance interposed between the electrode and reference ground. This construct results in multiple possible conduction pathways and therefore in low fidelity compared to a bipolar arrangement.² (tissue conductance in humans is highly variable not only geographically, but also temporally. In other words, tissue resistance might not only vary significantly from block to block of tissue, but also within each block as time progresses.

Very large arrays of these bipolar electrode pairs could be easily incorporated into thin flexible membranes in a manner analogous to the construction of large LCD television sets. Sensory stimuli are amenable to encoding in either analogue or digital format, identical to television signals. It is therefore envisioned that sensory broadcasts analogous to television broadcasts will eventually be commonplace. Applications may include, but would not be restricted to, the transmission of personal messages, general instruction, a further progression of remote and pre-packaged sexual propaganda (“pornography”), therapy designed to manage psychiatric disorders, therapy to reduce the perception of loneliness in elder care facilites and therapy intended to reduce the level of aggression in prison inmates. It would have applications in animal husbandry for the rearing of the young and reduction of aggression of adult livestock.

These data streams may be acquired and encoded in a raster type scanning pattern analogous to that used in generating and delivering television signals. It is quite probable that, given the significantly lower data density of tactile stimuli, current data transmission means would be much less taxed when used for the purpose of transmitting tactile data streams than streaming or broadcast video. It is thus anticipated that tactile stimuli can be acquired and coded at multiple loci simultaneously. This in turn would permit the delivery of signal along multiple loci simultaneously, resulting in higher perception fidelity.

Electronic sexual tactile stimulation devices would be a particularly useful and simple application. Small devices incorporating the above described features would be particularly appropriate for the purpose of stimulating oral-genital stimulation (“cunnilingus”) in the case of female subjects and anal/prostatic stimulation in the case of homosexual males. Due to the low spatial resolution of the erogenous zones and their susceptibility to desensitization to physical stimuli, even crudely constructed electronic stimulator arrays would have an advantage over a physical partner, as long as the the stimulating waveform is constructed with sufficient accuracy to result in the required quality of sensation. (i.e.: sinusoidal waveforms to generate a more indolent sensation, triangular and square waveform to generate a sense of urgency).

Appropriately formed devices to conform either to the introitus of the vagina or to fit inside the rectum and to provide sufficient rectal anatomy congruency to maintain contact with the prostate. A raster type stimulus delivery pattern would not be appropriate in such a setting, as human intercourse is not a mechanically repetitive or predictable activity. Mechanically repetitive and predictable activity is less effective and loses impact, as compared to real life activity. This is because the prime directive of any live animal (and particularly human beings) is to procreate and thus ensure the survival of the species. Specific neuroanatomic, as well as innate and learned behaviors contribute to recognize suitable partners and compel the animal towards sexual conduct leading to conception. Tactile and olfactory stimuli are central to this recognition and much more potent than sight or sound in initiating this behavior.

While not of comparable in fidelity to vision and sound, tactile and olfactory stimuli nevertheless these mechanisms do retain sufficient specificity that ensure that the animal copulates with a partner providing a reasonable chance of reproductive success. Optimal stimulus would thus have to be modeled on the real life activities of experienced and skilled sexual participants. Specific behaviors to be simulated would be simulation of the hand touch to erogenous zones, rotatory sweeping motion of an actual tongue and the insertion of the male member into desired orifices.

An obvious embodiment is envisioned to deliver simulated oral genital stimulus to the female vulva and clitoris. The bipolar delivery array would be contained within a suitably conformable matrix. Means of insertion into the introitus to the vagina may be provided. A power source and CPU would be integrated into the device and would generate a signal that would be distributed through the multiplexer to various points of this array. Manipulation of waveform, frequencies, voltages and currents would determine the quality of the sensation experienced by the subject. Sine wave, for instance causes a “soft” feel, versus an harsher one with triangular and, particularly, square waves.

The number of bipolar loci engaged and the speed of motion of the activated locations would determine the stage of excitement. Rapid motion would be more appropriate farther in the stimulation sequence and would lead to greater excitement. The same is the case with the number of loci activated at once, as well as the current intensity supplied to each locus.

Delivery of particular signal trains to individual locations, such as clitoris versus vaginal introitus, would permit simultaneous simulation of cunnilingus and penetration. It is envisioned that adequate experience with this technology will permit not only the subtle reproduction of existing interpersonal touch modalities, but also the generation of new sensory modalities. Specifically, manipulation of waveform shapes, as well as the variation of frequencies and the use of higher harmonics would color the sensory experience. The ability of share these experiences and specific electronic data is envisioned to enhance the extent of electronic interpersonal communications, as well as to permit the refinement of the fidelity of virtual reality environments.

Required frequencies are trivial (on the order of 100 Hz to 5 KHz) and even with the use of harmonics will certainly fall below 50 KHz. Since required voltages and current flows are also very low, the device would require a small power source and can thus be made very compact. The use of bipolar arrays would provide absolute control of the electrical discharge pathway. The use of a bipolar array is thus much safer than central ground and permits the use of the device anywhere on the body, including near the heart and including in people with implanted pacemakers and automatic implantable cardiac defibrillators (AICD). A bipolar array also permits much more precise coding and resolution than central ground. Finally, the use of software and hardware current limiters would provide an extra safeguard to ensure absolute security for the user.

All that being said, central grounding may be appropriate in certain settings. Bipolar arrays can be temporally defaulted to central ground, which would permit the coverage of large areas simultaneously. This is appropriate after a suitable period of build-up (“foreplay”), when nearing maximal sexual excitement (i.e.: orgasm). Direct remote user control (i.e.: not through the input port, but through independent wireless input, such as from a handheld wireless device) also is a consideration. Such a remote would provide command over signal strength, modulation of frequency, waveform and the distribution of the signal along the actuator electrode array. This would permit the user to tailor the stimulus to his/her individual preference.

While probably not necessary, the signal delivery component may integrate a feedback loop for sensing skin impedance to regulate output voltages. The feedback eventually may also include a sensor tied to variables such as heart rate to measure the level of arousal in the user. This would permit the routine to adjust the above mentioned variables automatically to deliver the maximally effective stimulus to the user.

Of note, much effort has been expended to emphasize the sexual potential of this technology. In part this is because an indirect effect of electronic female sexual stimulus might prove far more important. Female humans have been observe to possess an almost unlimited ability to respond to sexual stimulation, as long as desensitization is avoided. This sub-threshold stimulus in common parlance is known as “foreplay”. However, it is also known that even this vague level of arousal is capable of rasing the body temperature and thus the metabolic expenditure of the body. Chronic use of this stimulatory device is therefore likely to result in weight loss.

It is also known that adequate sexual stimulus has profound mood-altering effects on female humans. Sexual activity releases endorphins and endogenous serotonin and serotonin analogues. Use of this device may prove mood-altering in human females in a positive manner.

Additionally, provision of electronic self-stimulation means to homosexuals might result in reduction of high risk activity in the community. Such a modality is particularly likely to succeed given that homosexuals often have to resort to modalities of increasing intensity to achieve and maintain arousal. This is due to rapid desensitization induced by the often fierce activities they practice, as well as the extent of said activity, which inevitably results in tachyplaxis. As already alluded above in the body of this discussion, electrotactile stimuli do not exhibit tachyplaxis.

Finally, the current requirements for the proposed technology and embodiments are sufficiently low to permit very small physical dimensions for self-contained devices. Thus compacted, the devices may be worn at all times by the user. Particularly in the case of women, constant sexual stimulus leads to increased general arousal and metabolic activity without the side effects and morbidity resulting from drug or pharmacologic use. Weight loss application is thus envisioned for the technology.

### DISTINCTION FROM PRIOR ART

There are numerous patents proposing to capture various sensory stimuli and then to deliver them through a different sense. The majority of these patents address interconversion of visual information into tactile stimuli. While this may appear to be an extraordinarily sophisticated application (i.e.: “turning sight into touch”!), in fact it is not. This is because most patents deliver the tactile stimulus with only sufficient specificity to permit recognition of an individual stimulus, not to recreate the original sensation. USPTO #6430450 constitutes such an application and may serve as an example: tactile stimuli are delivered electronically, but the fidelity is only sufficient to recognize a pre-trained stimulus. It is expected that the user will be first trained by exposure to the requisite stimuli to be recognized; only after such training is the user assumed to be able to recognize these pre-conditioned stimuli. (i.e.: the user will be presented with what it “feels like” to “see” a flashing red light that would occur during a fire emergency; when the user senses this “sight” of a flashing red emergency light, he will recognize it and evacuate the building; of course, the user “senses” this sight through a particular sensation on the tongue, not through an accurate mental image of the flashing red light created by the actual flashing light).

There are no patents to propose the delivery of a real life tactile stimulus to a distant target with sufficient fidelity to trigger the same sensation in the recipient as if the original stimulus had been delivered directly. There are no patents or publications proposing the detection and analysis of vibrations for the purpose to reconstructing them electronically as an electronic stimulus for the purpose of generating a tactile sensation (i.e.: “touch from a distance”).

There are no patents or publications proposing to utilize Weber's Law to construct any sensory stimuli. There are no proposals in any patents for utilizing bipolar electrode arrays for the delivery of any stimulus. There are no means proposed of constructing any structures to deliver sufficient information density to replicate the sensation of touch (or warmth, or any other tactile sensation) to any degree of specificity at a skin target site. There are no means prescribed for parsing such information, whether whole array independently addressed, raster scanned, or any other method.

Several early patents (FR859618, USPTO # 1425743, 2622601, 3096768 & 3794022 ascribe nebulous therapeutic properties to low frequency electrical stimulus to the body as a whole (or a large subsegment) delivered from multiple sources in such a way that the signals “heterodyne” (i.e.: form an interference frequency). Aside from the fact that no therapeutic property has ever been demonstrated for such interventions, the proposed technologies are incapable of delivering high fidelity electronic tactile stimulus.

A myriad of prior endeavors are proposed for the generation of sexual stimuli to mirror experiences that would be realized with an active or passive real-life partner. There are no patents proposed to deliver a sexual stimulus through wholly electronic means (the electroejaculators and their analogs do not constitute a “sexual stimulus”, but rather elicit an ejaculation without the specific constellation of sensations together termed a “sexual experience”). There are no proposals to miniaturize any sexual device to an extent to permit continuous wearing of such a device. There are no means proposed to make a sexual device sufficiently innocuous to permit wearing in public without any possibility of detection.

While rather ingenious and cognizant of some aspects pertinent to the delivery of cutaneous stimuli, USPTO 6930590 describes a cumbersome and impractical device that would be complex to manufacture. In addition, none of the details required to achieve practicality are outlined. Weber's law is not mentioned. Finally, a bipolar array is not mentioned. Safety is an issue with central ground (centrally grounded apparatus cannot be used around the heart or in individuals with implanted pacemaker, automatic implantable cardiac defibrillators, or similar implanted electrical devices), as is the fact that it is not possible to achieve sufficient fidelity through central ground to achieve the illusion of virtual reality.

There are literally hundreds of patents for sexual equipment of various types, including electromechanical vibrators. Aside from their usually limited utility and at times pathologic applications, such devices are not applicable to the currently proposed technology and are therefore only presented through a limited number of representative patents (specifically, a limited number of vibrator patents).

Several “virtual reality simulators” are presented (USPTO # 5583478, 6326901 & 7271707). None provide any real-life data gathering means; they thus propose the delivery of ab initio synthesized data. Further, they all propose mechanical delivery of stimulus and are therefore only presented as background to the current proposal and to illustrate possible applications. None prescribe art that would contain means and methods that are contained in this patent, or would in any manner be applicable thereto.

USPTO #6430450 addresses a tactile output device intended to deliver tactile stimuli. Since the output is intended to be delivered into the mouth, and specifically into the tongue, the claims are not achievable. The tongue is a mixed tactile and gustatory device and any stimulus to the area would stimulate both senses. In addition, the intended target is not a high fidelity reproduction of tactile stimuli from a distance, but rather an alternate way of transmitting visual stimuli. There is thus no attempt made to replicate and modulate normal touch sensations, whether based on real life scenarios or synthesized ab initio.

USPTO #6430450 is incapable of reproducing high fidelity tactile stimuli since it does not specify a bipolar array. Further, it describes a preset scanning sequence (“raster scanning” or “outline scanning”). This is in contrast to the flexible system described in this patent application. A variable geometric stimulus pattern is particularly important to replicate the details of oral-genital stimulation, as described in the detailed description.

The multiplexer function outlined in USPTO #6430450 is also significantly different than in the current application. The multiplexer in USPTO #6430450 triggers an array of switches, instead of simply transmitting (“throughputting”) the stimulus signal. This adds unnecessary complexity and significant cost to the system.

The technology proposed in USPTO #6430450 is not intended to faithfully replicate tactile sensations and deliver them to a site that would be the original recipient. The tactile stimulus created has only sufficient specificity to permit recognition of an individual stimulus, not to recreate the original sensation. Tactile stimuli are indeed delivered electronically, but the fidelity is only sufficient to recognize a pre-trained stimulus. It is expected that the user will be exposed to the requisite stimuli to be recognized and after such training will be able to recognize these pre-conditioned stimuli. This patent contains sufficient restrictions (such as its intended target site being specifically the mouth and tongue) and omits sufficient further required technical skill to render it incapable of delivering high fidelity electronic tactile sensations.

USPTO # 5922012 describes an array intended to deliver muscle electrical stimulus. Electrodes are selectively paired within the array with the intent of avoiding desensitization due to the signal. This technology does not address active switching of signal with the intent of replicating a real world tactile stimulus, nor would it serve for the purpose of delivering high fidelity tactile stimulus to a recipient biological substrate. There is no purpose-built pre-structured integrated array specified to cooperate with the intended delivery site, nor is the information carried by the proposed system sufficient to even accidentally deliver a sensation that would be consistent with a real life touch sensation. Further, this is a deep-tissue application; the delivery of high-fidelity tactile stimuli requires confinement of the delivered electronic impulse to a sufficiently small and discrete physical area to be perceived as physically distinct from a neighboring stimulus.

USPTO # 6393325 prescribes a manner of calibrating a standard pre-formed array in terms of geometry and intensity of output. It does not address methods of construction of said array, bipolar arrangements, switching means, active switching of signal and is not intended (nor would it serve) for the purpose of delivering high fidelity tactile stimulus to a recipient biological substrate.

USPTO # 4124028 & 4564024 address the concept of electroejaculation. These are single channel devices that exploit the delivery of appropriately tuned electrical signals to result in ejaculation by a male of any desired mammalian species. In contrast to the currently proposed technology, the electroejaculation apparatus is remarkable for resulting in the emission of reproductive substances WITHOUT the specific sensations normally associated with such discharge. In that sense it is diametrically opposite to the current technology, which seeks to reproduce and even enhance real life sensations that would result from tactile stimulus (including erotic tactile stimulus), which may or may not lead to ejaculation.

USPTO # 4542753 describes an electrical prostate stimulator. It is basically analogous to the proposals above (USPTO # 4124028 & 4564024) in that its primary claims recommend delivery of an electrical signal to the prostate (and it is believed that these claims are in fact not subject to protection due to said duplication). There is some description of making various molds of anatomic structures, obviously not relevant to the currently proposed patent. It does not prescribe a structure of multiple electrodes, nor any controlled means of active switching signal to individual electrodes to restrict delivery of signal to a specific geometry and thus carry tactile information. The proposed technology is not intended (nor would it serve) for the purpose of delivering high fidelity tactile stimulus to a recipient biological substrate.

USPTO # 7006870 prescribes an implantable pudendal nerve stimulator. It does not include any geometric mapping, nor the ability to modulate the stimulus signal. It is not intended for, nor is it capable of delivering high fidelity tactile stimulus to the intended biological substrate.

### DESCRIPTION OF THE DRAWINGS

FIG. 1 illustrates the basic logic of the sensor apparatus. NOTE: sensor apparatus is optional (tactile signals can be generated de novo and/or ab initio). Items of interest are labeled on the diagram.

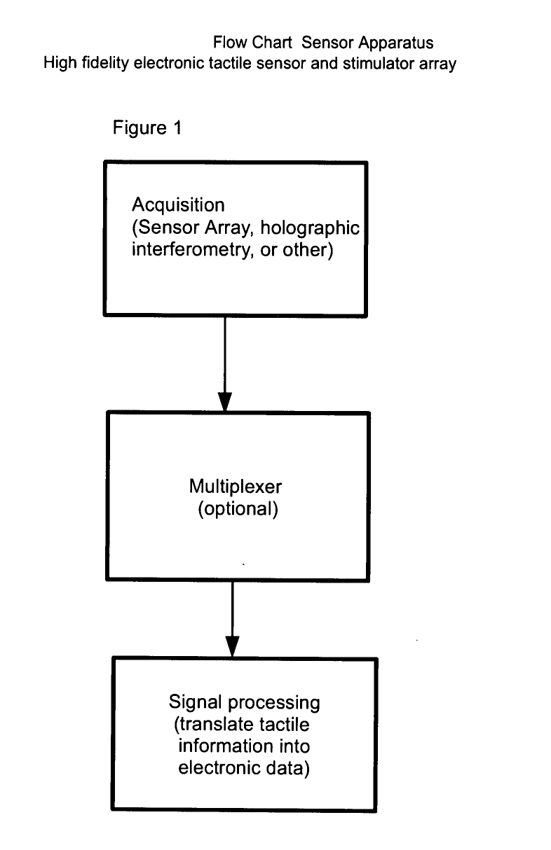

FIG. 2 illustrates the basic logic of the tactile delivery apparatus. NOTE: the CPU processes and/or synthesizes the electrical signals, parses the signals and controls the multiplexer to select delivery target. Items of interest are labeled on the diagram.

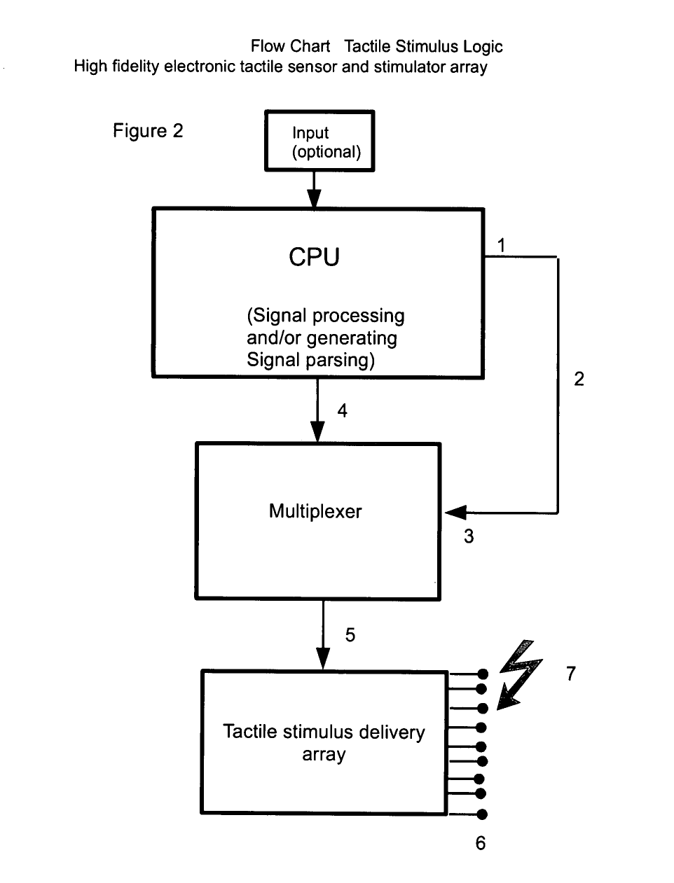

FIG. 3 illustrates the nature of a multiplexer controlled tactile stimulus delivery array. NOTE: in order to emphasize the primarily bipolar operation of the array, leads are physically paired. This may not be the case in actual operation (i.e.: lead 1a may operate paired with lead 4b; as long as such pairing does occur, however, the array is operating in a bipolar manner). Items of interest are labeled on the diagram.

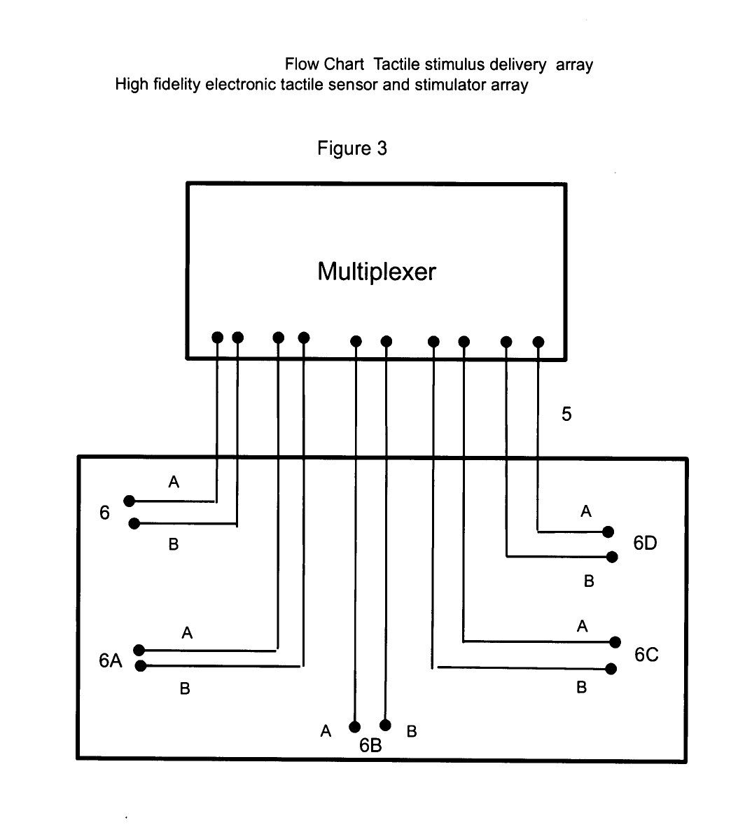

FIG. 4 illustrates a specific embodiment optimized for female sexual stimulus. The electrodes are embedded in an insulating matrix and are actuated either in a preset sequence stored in the CPU's memory, or governed by the input data. As this is a real world application, the contact electrodes are not physically paired up. However, they nevertheless do operate in a bipolar electrical configuration. Items of interest are labeled on the diagram.

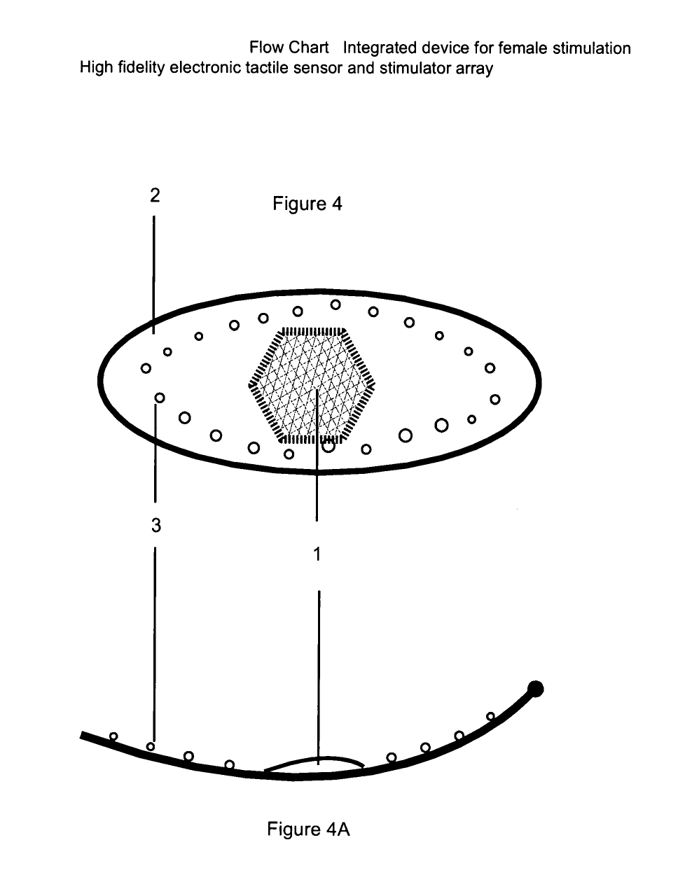

### DESCRIPTION OF THE SPECIFIC EMBODIMENTS

A compact self-contained device integrating an input port (a physical port versus wireless), coupled to a CPU, a multiplexer and a bipolar electrode delivery array to provide sexual stimulus either to the clitoris and vagina, or intra-rectally (separately shaped devices).

A complete system for the detection, analysis and transmission of tactile information to a distant bipolar electrode delivery array to convey the experience of a discrete tactile stimulus at a distance.

The transmission of virtual reality data streams via broadcast or internet to a distant bipolar actuator array in a manner analogous to the transmission of video signals today.

The integration of the capacity to accept and remotely transmit tactile stimuli into phones, computers and other basic electronic devices.

The provision of flexible sheets of bipolar electrode delivery arrays capable of providing remote touch stimuli for the purpose of stimulating people and animals for the purpose of behavior modification.

The enhancement of garments with bipolar electrode delivery arrays to augment the enjoyment of wearing said garments. The use of such arrays to inhibit or mask noxious stimuli, such as sensation of cold or heat.

One member of a couple separated by geographical distance kisses their mobile phone's display screen and the partner is able to sense this kiss through his/her mobile phone screen.

One member of a couple separated by geographical distance utilizes a computer interface and the internet (or a telephone with Bluetooth or other wireless capabilities) to remotely control the sexual stimulation apparatus described in this patent application and thus facilitate closeness and the illusion of participation in sexual intercourse.

1. Modular electronic and/or electro mechanical apparatus applicable to any species, including Homo Sapiens, for the detection, analysis, transmission and delivery of tactile stimuli

2. Detection and analysis functions of Apparatus of claim 1 presumed physically separate (heretofore called detection/analysis component)

3. The detection/analysis component of the Apparatus of claim 1 comprising some mechanism to detect vibratory stimuli within and on the surface of the target section of matter (embodiments to further clarify)

4. The detection/analysis component of the Apparatus of claim 1 capable of analyzing the detected information and resolving it into electronic signals that can be re-delivered to skin through a suitable electode delivery array to generate a tactile stimulus.

5. The detection/analysis component of the Apparatus of claim 1 possibly absent and replaced by other means of generation signal (such as de novo or ab initio signal synthesis) to be delivered by the delivery mechanism of the Apparatus of claim 1, thus exempting the invention from claims 3 and 4.

6. Standard or novel transmission means for the transmission of coded or analog electronic data to the signal delivery component of the Apparatus of claime 1.

7. The delivery component of the Apparatus of claim 1 comprising at least a power source, input component, processor unit, a signal distibutor and an electrode delivery array.

8. The input access of the delivery component of the Apparatus of claim 1 non-accessible to the user or accessible to the user through standard or non-standard means (detailed specifications to clarify), including physical connection and /or wireless means.

9. The electrode delivery array of the delivery component of the Apparatus of claim 1 to be integrated into a flexible matrix compatible with human and animal tissue and capable of conforming to the target site.

10. The electrode delivery array of the delivery component of the Apparatus of claim 1 capable of functioning in a bipolar manner with individual electrode pairs either functioning independently of each other, or cross referenced in a deliberately determined manner.

11. The electrode delivery array of the delivery component of the Apparatus of claime 1 capable of being secured through intrinsic or extrinsic means to the target site.

12. The Apparatus of claim 1 possibly provided with the means of remote control by the user or other party.

13. The optional provision of automatic feedback control integrated into the delivery component of the Apparatus of claim 1 premitting automatic control of impedance to maintain consistent output voltage and current.

14. The optional provision of automatic feedback control integrated into the delivery component of the Apparatus of claim 1 to detect arousal in the user and vary the signal generated for maximum impact on arousal at the particular level of arousal.

15. The optional provision of appropriate venues for users the Apparatus of claim 1 to collaborate in integrating information pertinent to control of said device, including details of receptive detection and analysis, transmission of information and delivery of said information.

16. The provision of means to obtain payment for data streams described in claim 15.

17. The application of the technology of claims 1-16 for the purpose of inducing sexual arousal, as well as to cause body weight loss through the induction of sexual arousal.

18. The numerical encoding of vibration information detected by any means utilizing Weber's law, individual biological touch receptor (i.e.: Meissner corpuscle, Merkel cell, Pacinian corpuscle, Ruffini nerve ending, free nerve ending, muscle spindle tension proprioceptor, temperature sensor and any other sensors known or not known that transduce primarily vibration modalities) transduction properties, as well as output characteristics, and weighting for local target touch receptor densities of each individual type to determine target signal parameters (frequency, amplitude, current, pulse sequences and length, parsing, and other parameters as deemed relevant) at individual array loci, or the use of analogous numerical encoding algorithms for ab initio synthesis of biological stimuli

19. The terms “biosense conde”, “biosensory code” and “biosensory encoding” to refer to the techniques of claim 18 above

20. The term “VYB” to represent the apparatus described by claims 1-19 of this patent and to be restricted from use in applications not consistent with these claims
